(19) United States
(12) Patent Application Publication
(10) Pub. No.: US 2002/0076698 A1
(43) Pub. Date: Jun. 20, 2002

(54) Gene reader: methods and apparatus for DNA identification and sequencing

(76) Inventor: Eric Orace Johnson, Champaign, IL (US)

Correspondence Address: MICHAEL BERNS, MALONEY, PARKINSON AND BERNS, 135 W MAIN STREET, URBANA, IL, 61801, US

(21) Appl. No.: 09/737,813

(22) Filed: Dec. 18, 2000

(51) Int. Cl. C12Q 1/68; C12M 1/34

(52) U.S. Cl. 435/6; 435/287.2

(57) ABSTRACT

An electronic microchip in possible conjunction with chemical tags for rapidly determining the sequence of genetic material. The tags are designed to facilitate automatic positioning and identification of genetic bases. The electronic microchip actively positions the subject material, senses the characteristics of the tags and transmits that information.

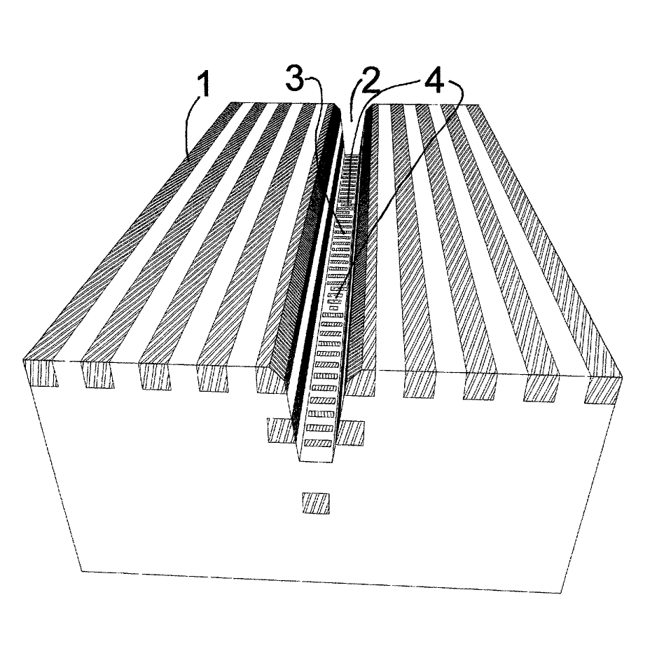

### BACKGROUND OF INVENTION

This invention relates to the transcription of sequences of genes and the structure of other molecules into computer readable data.

Currently, many firms are involved in discovering and transcribing DNA genomes. By knowing the exact sequence of base pairs in genomes, many advances in health care and plant sciences are possible. Frederick Sanger developed the basic chemistry in the 1970's.

In the chain termination method of DNA sequencing, a small portion of a DNA is replicated. The bio-chemical replication process includes fluorescent molecules that stop the replication process at random points. By using 4 different types of fluorescent molecules, the color indicates which base pair the molecule is replacing.

The assorted DNA portions are separated by chromatography into bands of differing molecular lengths. By looking at the sequence of colors, one can determine the sequence of base pairs in the whole DNA portion under study.

Problems:

Only small DNA portions (500 to 1000 base pairs) can be sequenced at a time. Determining the sequence of a whole genome takes a prohibitively long time and is costly.

In another method, Gene Chips probe for specific genes. An array of biochemical probes is deposited on a substrate. After chemical reaction, the color of each probe is measured to determine the result of each probe.

Problems:

Only a relatively small number of specific genes can be probed with this method.

SPM:

Scanning Probe Microscopes use charges, conduction or molecular forces along with atomic scale positioning to measure the shape of a surface. This has been suggested as a possible way to measure the different base pairs of a DNA molecule. There are many difficulties inherent in this proposition. Holding and positioning the helix of a DNA molecule while scanning it with a carbon nano-tube probe would be problematic and slow.

X-Ray Diffraction:

The DNA molecule is flexible so it doesn't form a good crystal. Instead, fibers of oriented DNA molecules must be used. But this fiber method does not provide good resolution. Even if a sufficient resolution could be achieved, the complexity of DNA requires that a huge number of x-ray images must be made and analyzed. Even with automated machinery and computer processing, it is a very time consuming process.

### SUMMARY OF INVENTION

In accordance with the present invention, a microelectronic circuit can more directly measure the characteristics of a molecule under study to identify it and determine the pattern of its components. The result is that complete gene sequences may be read rapidly at a minimal cost.

### BRIEF DESCRIPTION OF DRAWINGS

The FIGURE shows a perspective, cross section view of the relevant portion of one embodiment of the invention. The upper surface of the microelectronic chip is covered or layered with a multitude of conductive traces. The traces both move and sense the target molecule.

Long, parallel traces 1 are called aligners. They are used to move a target molecule radially or sideways to the “Sensing track” 2. Below and on the walls of the sensing track are more aligners. These aligners help orient the target molecule and hold it in the sensing track.

In the sensing track is a pattern of conductive elements. Some of these elements are know as ‘Movers’ 3. The movers can be used to electrostatically peristaltically move the target molecule axially along its long axis.

Other conductive elements are known as “Sensors” 4 and are used to sense the presence of specific molecules.

### DETAILED DESCRIPTION

Gene Reader design:

The invention has patterns of conductive traces connected to microelectronic circuits. The conductive traces are designed and positioned to allow for sensing and positioning of molecules. The microelectronic circuits control the probing forces and interpret the resulting signals. The microelectronic circuits also handle communication with external electronics.

Aligners:

One set of structures on the chip is designed to attract, straighten and/or channel the target molecule. If the target molecule, or portions of the target molecule have a consistent dipole direction, a plurality of wires can create a corresponding dipole moment which may be used to guide the target molecule to the desired location on the chip, straighten it and align it as desired.

The aligners may be long wires or segmented wire sets as show in the FIGURE. By using sets of wires, the aligners can provide a twisting force to the target molecule. This force can be used to straighten the DNA helix.

The aligners can be used to deform the target molecule. By using long, parallel aligners, long and straight channels of electrostatic potential are formed. The dipole moment of the target molecule will force the target molecule to straighten to the shape of the aligners dipole field.

The aligners may be used to untwist a DNA molecule. By segmenting the aligners, making an array of them and driving them with the proper phase relationships, one end of the DNA can be rotated in a direction that is not matched by the other end. This can be used to twist the DNA to a precisely determined helix or to completely straighten the DNA, simplifying the processes of moving it and reading it's bases.

Movers:

Zones on the gene reader chip can use charges, currents, magnetic forces, mechanical probes and/or optical forces to move and bend the molecules into various desired alignments and positions. These zones can be at different depths under the chip surface, on or near the chip surface and above the surface.

Small charged areas in close proximity to the target molecule's substructures will be used for small movements and to lock the target in place so it may be measured. When used together, the charged areas function as the stator in a linear motor designed to move the target molecule along the desired path. The target molecule may be moved as a stiff unit or flexed peristaltically in a fashion similar to the movement of a caterpillar. The distance between individual stator elements and the distance between the repeating elements of the target molecule can fall into a wide variety of ratios. By using a larger stator separation, production of the chip is simplified. When construction techniques can produce smaller stator elements, a much smaller distance between each of them can allow more precise positioning of the target molecule.

Target molecules can also be moved by creating a surrounding magnetic field and then driving a chosen amount of charge through a conductive portion of the target. The resulting force will move the target a precise distance.

Sensors:

Conductive areas can transmit electrostatic and magnetic forces to the substructures of the target molecule. The target will then alter those forces and/or produce other forces that will be measured by structures on the chip. For example: The rate at which a conducting area on the chip can be charged is affected by the presence of the dipole field of the target molecule. This can be used to determine not only the presence of a target molecule, but also what type of target molecule substructure is adjacent to the sensor.

There are several ways to measure a dipole moment. In the dielectric dispersion measurement technique, an oscillating current is placed on a plate above the chip and on the sensor wires in the chip. The voltage measured on the plate and wires is lower if a dipole (the tag) is between the plate and the sensor.

In the fluorescent emission measurement technique, the emission of some wavelengths can be dependent upon the tag having a dipole moment. Measurement of precise colors emitted under different conditions can then be used to detect and differentiate dipole tags.

Multiple Sensors:

The chip can have a multitude of sensors (4). This allows for error checking and faster determination of the molecule being observed. The elements used for sensing and positioning of the target molecule can operate simultaneously or sequentially. Different sensors can be tailored to respond to different tags.

Tags:

Since the outside of a DNA helix is uniform, it is hard to measure directly. By splitting the chain open and attaching readily identifiable molecules to the bases, the gene reader chip can easily identify the corresponding structure of DNA bases.

Tags may be designed to mate with individual bases, or to larger sequences of bases. By mating with several bases, the measurable portion of the tag may be made larger. While more tag types will be needed, this makes design and manufacture of the gene reader chip simpler.

Handles: Molecules can be attached to the target which allow the gene reader chip to readily move, position and hold the target molecule in place. The same molecules can be used as both tags and handles. A combination of the structures that naturally occur in the target molecule and added structures may be used for tags and handles.

The tags and handles can also be designed to change the shape and stiffness of the resulting target molecule. For instance, by adding molecules that link with each other, the spiral of a DNA helix can be straightened and a new, stiff backbone created.

This will make it simpler for the movers and sensors to transport and measure the target molecule.

Chip Microstructure:

The physical shape of the gene reader chip may be designed to facilitate the smooth passage and containment of the target molecule. An appropriately sized groove with funnel shaped entrance and exit will help guide the molecule. If the chip is to measure a helix molecule, the groove can be spiral shaped or a series of parallel grooves that mesh with the twists of the helix.

Chip Layers:

Structures to sense and/or move the tags can be on the surface, buried in, below or suspended above the chip.

Sequential Addition of Tags:

Since the mating surfaces of the tags are designed to adhere to complimentary base pairs, the tags themselves can adhere to each other. To prevent complications from this effect, 2 non-complimentary tag types can be allowed to adhere to the split DNA strand. After all the relevant DNA bases have been tagged, the other tag types may be added. This limits the waste of tags while properly tagging the target molecule.

Measurement in Parts:

The target molecule may be measured in several passes. During the first pass, a tag for one base or set of bases is used. The gene reader chip notes either the presence or absence of a tag and its type at each position along the target molecule. Subsequently, copies of the target molecule are tagged to reveal the other possible bases or base groups. The multiple readings are then put together to reveal the complete sequence of the target molecule. The advantage of this method is that the wide varieties of tags do not have to be distinguishable from each other. Each tag can be larger and more easily read. In each pass, 1 or more distinguishable tag types may be used. This can speed up the reading process and allow the multiple readings to be easily reassembled into a single, complete sequence.

Mutations:

Since the bases in a DNA can mutate, special tags can be designed which mate with the mutated bases and convey that information to the sensors on the gene reader chip.

Design of the Tags:

Tags can be made in a wide variety of shapes and formulas. One method is to make parallel layers of carbon rings. The rings in one layer will be double bonded while the rings on the other layer will have their carbons terminated in hydrogen atoms. This results in a flat, rigid structure with a measurable electronic dipole. The tag's dipole moment allows it to be positioned by the gene reader chip. The dipole strength of each tag can be individually tailored. By measurement of the dipole potential and position, the identity of the tag may also be determined.

These tags may be made in differing sizes. The width and length of tags can be used to identify them. Both symmetrical and non-symmetrical tags may be used. Since the width of a tag affects both the position and the strength of its dipole moment, the sensors can measure these characteristics to determine which tag is being sensed.

Direct optical measurements may be also be used. Both frequency and intensity can identify each given tag. Optical piping and high frequencies (including x-rays) of light can facilitate this method. Optical methods may be used in conjunction with dipole measurement and other methods. Optically active tags can be designed. For example: bonding or caging a crystalline silicon nano-particle to the tag will result in a molecule with an ultra-bright fluorescence.

Direct Measurement:

If the structures on the gene reader chip are made fine enough and the target molecule has the appropriate characteristics, the target molecule and its micro-details may be determined directly without the use of tags and/or handles.

Chip Construction Techniques:

Since the structures on this chip are extremely small, some indication of a method of construction needs to be given.

One method is to deposit a conductive film on a substrate. A fine probe such as an atomic force microscope probe may be used to carve the film, creating conductive and insulating areas. An atomic force microscope may also be used to pick up individual atoms and place them in desired spots. A layer of insulating material may then be used to smooth the resulting surface. Additional layers of conductive and/or insulating materials may be applied to create all the sensing and controlling zones as well as the desired physical shape of the “gene reader chip”.

Operation:

A sample of cells is placed in a cuvet. A lyseing fluid is added and/or the cells are washed over a series of spikes to release the DNA. The DNA leaks out and is separated from the cells' membranes. In an appropriate sequence and timing, other chemicals are added to split the DNA helix and add the tags and handles. As the tag molecules are allowed to bond with the single helix strand, a readable molecule is formed. The DNA molecules are then transported to and positioned on an electronic chip, “the gene reader”. The chip uses aligners to move, untwist and position the target molecule along its reading section. The chip then sends pulses to its sensors, measures the responses and determines which tag is present at each sensor. After reading a tag, the chip pushes and pulls the molecule along so the next tag is positioned for reading. This continues until the whole desired section of the molecule has been determined.

A whole chromosome may be sequenced in this fashion. A plurality of sensing tracks can allow many different chromosomes may be read at the same time.

The gene reader chip and external computers assemble the data into the correct form and order.

Physical Guide Channel:

The FIGURE shows the microchip with a physical channel for the DNA to follow.

Other Embodiments—Electrostatic Guide Channel:

An electrostatic channel for the target molecule can be created from wires which create appropriate force gradients.

Other Embodiments—Simple Construction:

The chip may be designed for simpler construction with as few layers as possible (3)—horizontal conductors, insulator, vertical conductors.

Other Embodiments—Sensor Shapes:

The sensors may be designed to have a physical structure that partially mates with the DNA bases to be measured. By meshing with the target molecule, the sensors can give a highly accurate measurement of complex molecular structures. This process in effect forms the tags directly into the sensor arrays.

Other Embodiments—Projectile Sensing:

A sensor can be designed that operates by propelling an atom or group of atoms at the molecule. The atom bounces off the target molecule with a distinguishable direction, energy and charge. The sensors measure the presence of the resulting projectile to determine the structure of the target molecule.

Conclusion, Ramifications, and Scope:

This process can be used for DNA, RNA and many other molecules.

Speed:

Much, much faster than chemical and diffraction methods.

Accuracy:

It not only identifies genes and other groups of base pair sequences (like the chemical methods), but also creates a complete sequence map of the whole molecule.

1] A device for determining the structure of a molecule, comprising:
a means for sensing characteristics of portions of a target molecule or molecules and a means for communicating information whereby the structure of a molecule may be determined and disseminated.

2] The device of claim 1, wherein the means of sensing utilizes electrostatic forces whereby the molecule's characteristics may be sensed.

3] The device of claim 1, wherein the means of sensing utilizes conductivity whereby the molecule's characteristics may be sensed.

4] The device of claim 1, wherein the means of sensing utilizes mechanical probes to measure the shape of the molecule whereby the molecule's characteristics may be sensed.

5] The device of claim 1, wherein the means of sensing utilizes optical measurements whereby the molecule's characteristics may be sensed.

6] The device of claim 1, further including a means for movement and positioning of a target molecule or molecules whereby the desired portions of the molecule may be positioned for study.

7] The device of claim 6, wherein the means of positioning utilizes electrostatic forces whereby the molecule may be moved and/or held in position.

8] The device of claim 6, wherein the means of positioning utilizes mechanical forces whereby the molecule may be moved and/or held in position.

9] The device of claim 6, wherein the means of positioning utilizes optical forces whereby the molecule may be moved and/or held in position.

10] The device of claim 1, further including a means for interpreting the sensed characteristics of said target molecule or molecules whereby the raw data signals are converted into more useful forms.

11] The device of claim 1, further including a means for receiving instructions and information from external sources whereby it can be controlled.

12] The device of claim 1, further including atoms to be linked to the target molecule's structure whereby the movement of a molecule may be facilitated.

13] The device of claim 1, further including atoms to be linked to the target molecule's structure whereby sensing of characteristics of portions of a molecule's structure may be facilitated.

14] A method for determining the structure of a molecule, comprising the steps of:
positioning a molecule adjacent to a device, sensing the characteristics of atoms or atomic groups within the said molecule which is adjacent to the device, if necessary, repositioning the said molecule so other atoms or atomic groups may be sensed and repeating the process whereby the all the desired information about the molecule will be determined.

15] The method of claim 14, further including a step of outputting the resulting data whereby the distinguishing characteristics of said molecule may be disseminated.

16] The method of claim 14, further including a step of linking atoms to the target molecule whereby the movement, positioning and/or sensing of the target molecule may be facilitated.

17] A device for positioning a molecule, comprising:
a means for movement of a target molecule or molecules and a means for holding said molecule or molecules in a desired position whereby a molecule may be positioned so as to allow other operations to take place on it.

18] The device of claim 17, further including a means for communicating with the device whereby external control may be applied to the process.

19] The device of claim 17, further including atoms to be linked to the target molecule's structure whereby sensing of characteristics of portions of a molecule's structure may be facilitated and/or the movement of a molecule may be facilitated.
